(19) United States
(12) Patent Application Publication
(10) Pub. No.: US 2008/0306581 A1
(43) Pub. Date: Dec. 11, 2008

(54) Streamlined Stents

(75) Inventors: Joseph Berglund, Santa Rosa, CA (US); Susan Rea, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/759,420

(22) Filed: Jun. 7, 2007

(51) Int. Cl. A61F 2/06

(52) U.S. Cl. 623/1.15

(57) ABSTRACT

A stent for implantation within the body of a patient is disclosed. The stent can be formed from one or more stent modules comprising a plurality of stent struts, one or more of which have an inner contour designed for streamlined fluid flow when the stent is implanted within an anatomical passageway of the patient.

### FIELD OF THE INVENTION

The present invention relates to streamlined designs and configurations for stents that can reduce fluid flow disruption and decrease or eliminate areas conducive to material build up.

### BACKGROUND OF THE INVENTION

Cardiovascular disease, including atherosclerosis, is the leading cause of death in the United States. The medical community has developed a number of methods and devices for treating coronary heart disease, some of which are specifically designed to treat the complications resulting from atherosclerosis and other forms of coronary arterial narrowing.

One method for treating atherosclerosis and other forms of coronary narrowing is percutaneous transluminal coronary angioplasty, commonly referred to as “angioplasty” or “PTCA”. The objective of angioplasty is to enlarge the anatomical passageway of an affected coronary artery by radial hydraulic expansion. The procedure can be accomplished by inflating a balloon within the narrowed passageway of the affected artery. Radial expansion of the coronary artery can occur in several different dimensions, and can be related to the nature of the plaque. Soft, fatty plaque deposits can be flattened by the balloon, while hardened deposits can be cracked and split to enlarge the passageway. The wall of the artery itself can also be stretched when the balloon is inflated.

Unfortunately, while the affected artery can be enlarged, in some instances the passageway narrows again (“restenoses”), or closes down acutely, negating the positive effect of the angioplasty procedure. In the past, such restenosis has frequently necessitated repeat angioplasty or even open heart surgery. While such restenosis does not occur in the majority of cases, it occurs frequently enough that such complications comprise a significant percentage of the overall failures of the angioplasty procedure.

To lessen the risk of restenosis, various devices have been proposed for mechanically keeping the affected passageway open after completion of the angioplasty procedure. Such endoprostheses (generally referred to as “stents”), are typically inserted into the anatomical passageway, positioned across the lesion or stenosis, and then expanded to keep the passageway clear. The stent overcomes the natural tendency of the passageway walls of some patients to restenose, thus maintaining the patency of the passageway.

Stents can be formed using any of a number of different methods. For example, one method is winding a wire around a mandrel, welding or otherwise forming the wire into a desired stent configuration. A second method is by machining tubing or solid stock material into bands, and then deforming the bands into a desired stent configuration. Additional methods include laser etching or chemical etching tubes into the desired shapes.

A drawback of these manufacturing methods is that the inner surface of stent struts (struts are structural portions that together form a stent) is generally not sufficiently streamlined to avoid certain side effects of the stent. For example, when the inner surface of a stent strut is substantially planar and has abrupt edges along its periphery, turbulence can be introduced into the blood flow. Additionally, the abrupt edges can provide sites where plaque and other deposits can collect, which can lead to narrowing of the passageway and restriction of blood flow.

In order to reduce or eliminate these and other undesired side effects of a deployed stent, it would be desirable to provide a stent with struts that have streamlined inner contours. The present invention addresses these needs, among others.

### BRIEF SUMMARY OF THE INVENTION

In general terms, the present invention is directed to stents that can reduce or eliminate fluid flow disturbance once deployed at a treatment site and/or reduce or eliminate areas conducive to plaque or other material build-up. This is accomplished by, among other things, providing stent struts with streamlined contours to the treatment site, which may be, e.g., a constriction or narrowing in a blood vessel.

One embodiment of the present invention is a stent having a plurality of stent struts forming at least one stent module. In one embodiment, the stent module defines a passageway. In another embodiment, at least one of the plurality of stent struts has a streamlined inner surface.

In another embodiment of the present invention, at least one of the plurality of the stent struts has an inner leading surface and an inner trailing surface that are asymmetrical.

In another embodiment, the inner leading surface has an inner leading edge, the shape of which is defined by an inner leading curve. In another embodiment, the inner trailing surface has an inner trailing edge, the shape of which is defined by an inner trailing curve. In yet another embodiment, the radius of the inner leading curve is smaller than the radius of the inner trailing curve.

In another specific embodiment, the inner trailing surface is tapered. In yet another specific embodiment, the inner surface of the stent modules has an airfoil contour or a teardrop contour.

In another embodiment, at least one of the plurality of stent struts further comprises an outer surface generally opposite of the inner surface. In another embodiment, the outer surface comprises an outer leading surface and an outer trailing surface. In yet another embodiment, the outer leading surface and/or the outer trailing surface are chamfered or beveled. In yet another embodiment, the outer surface and inner surface have substantially similar or identical contours.

In yet another embodiment of the present invention, the stent is formed from metals, alloys, biologically compatible polymers, or combinations thereof. In one specific embodiment, the inner surface of the stent modules is formed from biologically compatible polymers.

In yet another embodiment, the stent comprises a plurality of stent modules.

The present invention also provides methods for treating the narrowing or constriction of an anatomical passageway by positioning at the narrowing or constriction a stent according to any of the herein-described embodiments.

Other objects, features, and advantages of the present invention will become apparent from a consideration of the following detailed description.

### BRIEF DESCRIPTION OF THE DRAWINGS

FIG. 1 depicts an exemplary stent module configuration that can be used as a portion of a stent body in accordance with the present invention;

FIG. 2 depicts a stent in accordance with an embodiment of the present invention implanted in an anatomical passageway; and

FIG. 3 depicts a cut-away cross-sectional view of stent struts deployed in a fluid passageway. The stent struts have substantially similar inner and outer curve contours in accordance with one embodiment of the present invention.

### DEFINITION OF TERMS

As used herein, “animal” shall include mammals, fish, reptiles and birds. Mammals include, but are not limited to, primates (including, without limitation, humans), dogs, cats, goats, sheep, rabbits, pigs, horses and cows.

As used herein, “drug(s)” shall include any compound or bioactive agent having a therapeutic effect in an animal. Exemplary, non limiting examples include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. Ser. No. 10/930,487) and zotarolimus (ABT-578; see U.S. Pat. Nos. 6,015,815 and 6,329,386). Additionally, and other rapamycin hydroxyesters as disclosed in U.S. Pat. No. 5,362,718 may be used in the present invention.

As used herein, “streamlined” means designed or arranged to reduce resistance to or interruption of fluid flow relative to the resistance to or interruption of fluid flow created by more planar surfaces or surfaces with edges that are more abrupt.

As used herein, “therapeutic effect” means an effect resulting from the treatment of an animal that alters (e.g., improves or ameliorates) the symptoms of a disease or condition, or the structure or function of the body of the animal; or that cures a disease or condition.

### DETAILED DESCRIPTION OF THE INVENTION

As stated, a drawback of previously used stents is that the stents are generally not sufficiently streamlined to avoid certain side effects. For example, when the inner surface of a stent strut is substantially planar and has abrupt edges along its border, turbulence can be introduced into fluid flow (e.g., blood flow). Additionally, stent struts with abrupt edges can create sites conducive to plaque and other deposits build-up, which can lead to narrowing of the anatomical passageway and restriction of fluid flow. The present invention is directed to stents that minimize the disturbance of fluid flow and/or reduce the presence of areas conducive to plaque and other deposits build-up in an anatomical passageway. Stents of the present invention provide this benefit by incorporating stent struts with streamlined contours.

Any balloon-expandable stent can be used as a stent in accordance with the present invention. For non-limiting examples, see United States patent numbers (“USPN”) U.S. Pat. No. 5,292,331 to Boneau, U.S. Pat. No. 5,135,536 to Hilstead, U.S. Pat. No. 5,158,548 to Lau et al., U.S. Pat. No. 4,886,062 to Wiktor, and the references cited therein. The present invention is applicable to all known stent configurations, and it will be readily apparent from the following discussion of several exemplary configurations how the invention can be applied to any other type of stent construction.

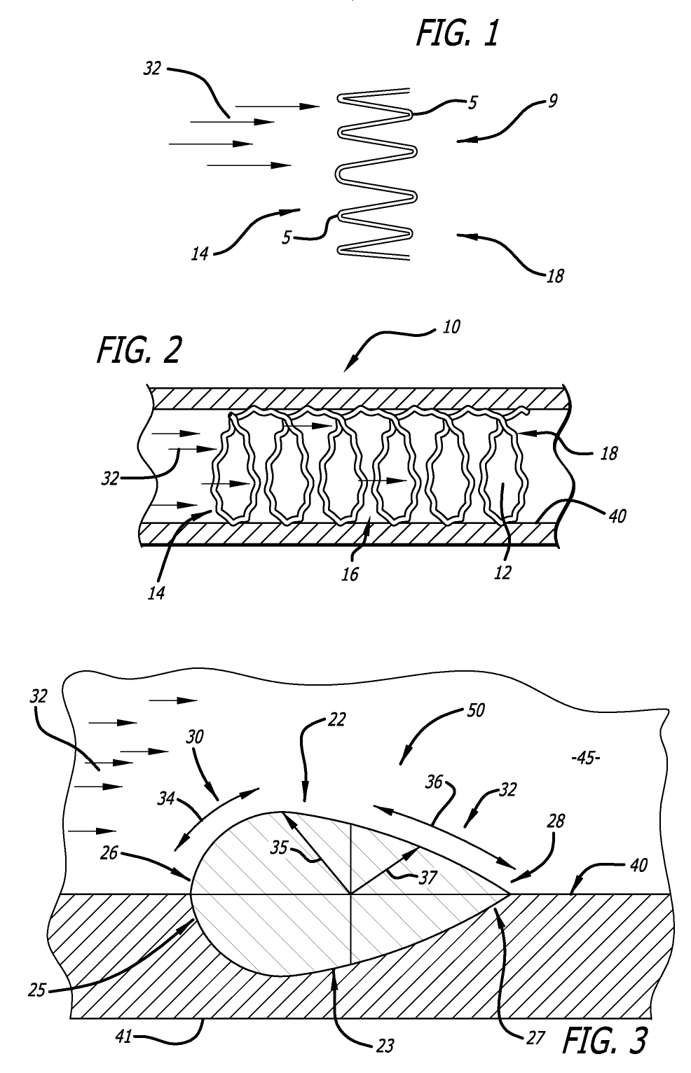

FIG. 1 depicts an exemplary stent section or “module” configuration useful in the present invention. In the illustrated embodiment, module 9 includes a plurality of struts 5 forming a zigzag pattern, although a skilled artisan will appreciate that many different modules and configurations can be used. Fluid 32 flows into stent module 9 (or a plurality of stent modules that form a stent) from the leading end 14 of the module and out of the trailing end 18 of the module.

A complete stent body structure can be formed from one or more stent modules (like the one depicted in FIG. 1) that include roughly circular groupings of stent struts 5. The stent struts 5 have an inner surface, which will be exposed to fluid flow when the stent is deployed. The inner surface of the stent struts will have streamlined contours that reduce or eliminate the disruption of fluid flow through the hollow channel of the stent and/or the provision of sites conducive to the deposition of materials (e.g., plaque, blood clots) or the growth of tissue within the hollow channel of the stent when the stent is implanted in an anatomical passageway. The stent struts 5 also have an outer surface opposite the inner surface. As known by the skilled artisan, the outer surface of a stent strut rests against (or is embedded into) the walls or inner surface of the anatomical passageway when the stent is deployed.

Referring now to FIG. 2, an alternative stent 10 is shown implanted within an anatomical passageway, which is defined by inner wall 40 of that passageway. As shown, stent 10 can be cylindrical or tubular in shape and can have a leading end 14, a midsection 16, and a trailing end 18. As discussed above, a plurality of stent modules may make up a stent. Additionally, the hollow channel 12 extends longitudinally through the body structure of stent 10. The structure of stent 10 allows for its insertion into the passageway defined by the inner walls 40. Once deployed, stent 10 physically holds open the anatomical passageway by exerting a radially outward-extending force against inner walls 40 of the passageway. Stent 10 also may expand the opening of the anatomical passageway to a diameter greater than the anatomical passageway's pre-implantation diameter and, thereby, may increase fluid flow through the passageway. As shown in FIG. 2, fluid 32 (e.g., blood, bodily fluid, etc.) flows through hollow channel 12 of stent 10 after the stent is implanted within the passageway defined by inner the walls 40.

FIG. 3 shows a cut-away cross-section view of a stent strut 50 embedded in the inner walls 40 of an anatomical passageway 41. While this figure depicts about half of the struts' cross-sectional area embedded into the inner walls 40 of the anatomical passageway, a skilled artisan will appreciate that this is not intended to limit the invention. As shown in FIG. 3, strut 50 has inner surface 22 made up of inner leading surface 26 and inner trailing surface 28. Strut 50 also has outer surface 23 made up of outer leading surface 25 and outer trailing surface 27. Inner leading surface 26 includes inner leading edge 30 and the trailing surface 28 includes inner trailing edge 32. The inner leading edge 30 and the inner trailing edge 32 are defined by inner leading curve 34 and inner trailing curve 36, respectively. Inner leading curve 34 and the inner trailing curve 36 each have radii of curvature 35 and 37, respectively, such that inner surface 22 can be provided with a streamlined contour. Of course, outer surface 23 of stent strut 50 may have the same shape as, a similar shape to, or entirely different shape than, inner surface 22.

In one embodiment, inner surface 22 may be provided with an asymmetrical contour, i.e., one where inner leading surface 26 and inner trailing surface 28 have, among other possibilities, different inner leading curve 34 and inner trailing curve 36, respectively. In another embodiment, inner leading curve 34 may be provided with a larger radius of curvature than inner trailing curve 36. The radius of curvature is measured from the “center” of strut 50 to the surface (inner or outer) of strut 50. For example, in the Y-plane in FIG. 3, the center is halfway between the inner and outer diameters of a stent (i.e., halfway between inner surface 22 and outer surface 23). In the X-plane in FIG. 3, the center is halfway between the front and back of the strut. The radius of curvature is the length from the center of the strut to the edge of the strut. As the skilled artisan will appreciate, both radii of curvature measurements must be conducted at equal angles (absolute value) from the Y-axis or the X-axis. In FIG. 3, radius of curvature 35 and radius of curvature 37 were both measured at about 45 degrees from the Y-axis (and X-axis). As the skilled artisan will also appreciate, radii of curvature 35 and 37 measured at 0 degrees from the X- or Y-axis (or 90 degrees from the X- or Y-axis) will be equal based on the definition of “center”. As depicted, the resulting strut cross-section resembles that of a wing or airfoil. While not required by the present invention, FIG. 3 depicts a stent strut with substantially similar inner surface 22 and outer surface 23 such that the resulting strut cross-section resembles that of a wing or airfoil. The asymmetry in contours between inner surfaces 26 and 28 of strut 50 can be beneficial when the stent is implanted such that the inner surface with the larger radius of curvature (here, inner leading surface 26) is oriented to face directly into or against the flow of fluid 32 (i.e., where inner leading surface 26 is upstream of inner trailing surface 28).

Those skilled in the art will appreciate that the inner leading edge 30 and/or the inner trailing edge 32 can have a variety of shapes and/or sizes that provide inner leading and trailing surfaces 26 and 28 of inner surface 22 with a streamlined contour (e.g., a teardrop-shaped contour, a airfoil-shaped contour, etc.). Many other shapes, surface lengths, curves and angles can be utilized to produce a stent/strut optimized for beneficial fluid flow patterns and manufacturing ease. Design optimization may be carried out by, for example, computational fluid dynamic studies. It can also be appreciated that in certain embodiments inner surface 22 may be very smooth (e.g., polished) in order to further decrease fluid drag in passageway 45 of the stent.

As shown in FIG. 3, outer surface 23 of stent strut 50 may be configured to embed into inner wall 40 of anatomical passageway 45 when the stent is deployed. Outer surface 23 may also be defined by outer leading surface 25 and outer trailing surface 27 (with respective outer leading and trailing curves and radii of curvature). As shown in FIG. 3, outer leading surface 25 and/or outer trailing surface 27 may have contours that are similar or identical to inner surfaces 26 and 28. Alternatively, the outer surfaces may have chamfered or beveled edges. As a skilled artisan would appreciate, the inner and outer surfaces of the stent struts can be designed in a number of configurations balancing ease/cost of manufacture with ease of the stent deployment and reduction of fluid flow interference. U.S. patent application Ser. No. 10/107,473 (published as U.S. 2003/0187498), the entirety of which is incorporated herein by reference, describes stent strut outer surface design. In another embodiment, the outer surface 23 of stent strut 50 can be provided with a smooth surface that can be beneficial in reducing the injury to and/or inflammation of the wall or inner surface 40 when a stent is implanted in an anatomical passageway.

Those skilled in the art will appreciate that stents of the present invention may be manufactured with a variety of shapes and/or orientations provided that laminarity of fluid flow is increased. Furthermore, any number of stent modules and/or stent struts may be joined or coupled depending on the physiological constraints of the patient. In one embodiment, any number of stent modules and/or stent struts of equal length and/or diameter may be coupled together to form the stent. In an alternate embodiment, any number of stent modules and/or stent struts of unequal length and/or diameter may be coupled together to form the stent. Furthermore, stent modules and/or stent struts can be manufactured from the same or different materials to produce stents. In another embodiment, the streamlined stent struts of the present invention may be coated with drugs to produce drug-eluting stents in accordance with the present invention.

Stents according to the present invention can be fabricated from any of several methods known to those skilled in the art. For example and without limitation, laser cutting a pattern in a tube, chemical etching a pattern in a tube, electron discharge machining a pattern in a tube, or wire extrusion can be utilized. The manner and shape of stents of the present invention can be numerous and can be made from a tubular segment or alternatively shaped with wire or wire-like meshing. The stent struts may be provided with shapes in accordance with the present invention (e.g., airfoil) by mechanical abrasion. Sand-blasting is one form of mechanical abrasion and may be carried out with small hard particles and/or high pressure fluid, whereby the particles and/or fluid flow over the stent strut in the same direction blood would flow over the stent strut upon implantation in a blood vessel. The sand-blasting process would wear away the areas of the stent struts with the highest resistance to flow first, thereby leaving a streamlined contour in accordance with the present invention.

Stents according to the present invention can be manufactured from a plurality of materials, including, without limitation, stainless steel, tantalum, titanium, nickel-titanium alloys, shape memory alloys, super elastic alloys, low-modulus Ti—Nb—Zr alloys, cobalt-nickel alloy steel (MP-35N), various biologically compatible polymers (including, without limitation, bioabsorbable, biodegradable and/or bioerodable polymer material or a biostable polymer material) and elastomers, including non-porous, porous, and microporous polymers or elastomers. In one embodiment, stents according to the present invention can be coated with or have applied thereto at least one drug, thereby enabling the stents to elute or deliver at least one drug to target site within the body of a patient.

Stents according to the present invention can also be provided with a polymer coating. Polymer coatings can be useful in increasing bare-metal stent biocompatibility and/or in serving as reservoirs for eluteable drugs. When used as a drug delivery platform, the coating of the present invention can be combined with a drug in a fashion optimally suited to deliver the drug, or drugs, over a predetermined time and with a particular kinetic profile. Polymer coatings can be applied to at least one surface of stent struts in a variety of ways, including, without limitation, dip, spray, or vapor deposition, or by other methods known to those of ordinary skill in the art. In one embodiment, polymer coatings can be applied to the inner surface of the stent struts to provide and/or augment a streamlined contour for the inner surface. Again, polymer coatings can include, without limitation, bioabsorbable, biodegradable and/or bioerodable polymer material or a biostable polymer material.

Many different polymers are known to be useful in accordance with the teachings of the present invention and can include, without limitation, poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen, hyaluronic acid, poly-N-alkylacrylamides, poly depsi-peptide carbonate, polyethylene-oxide based polyesters, polyurethanes, silicones, polyesters, polyolefins, polyisobutylene, ethylene-alphaolefin copolymers, acrylic polymers, acrylic copolymers, ethylene-co-vinylacetate, polybutylmethacrylate, vinyl halide polymers, vinyl halide copolymers, polyvinyl chloride, polyvinyl ethers, polyvinyl methyl ether, polyvinylidene halides, polyvinylidene fluoride, polyvinylidene chloride, polyacrylonitrile, polyvinyl ketones, polyvinyl aromatics, polystyrene, polyvinyl esters, polyvinyl acetate, copolymers of vinyl monomers with each other and olefins, ethylene-methyl methacrylate copolymers, acrylonitrile-styrene copolymers, ABS resins, ethylene-vinyl acetate copolymers, polyamides, Nylon 66, polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, or various combinations thereof.

The terms “a,” “an,” “the”, and similar referents should be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended to better illuminate embodiments according to the invention

Groupings of alternative elements or embodiments according to the invention disclosed herein are not to be construed as limitations. Each group member may be referred to individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description requirement for any Markush group used in the claims.

Many embodiments of this invention have been described. Of course, variations of these embodiments will become apparent to those of ordinary skill in the art upon reading this description unless otherwise indicated herein or otherwise clearly contradicted by context.

Furthermore, numerous references have been made to patents and printed publications throughout this specification. Each of the cited references and printed publications individually are incorporated by reference herein in their entirety.

What is claimed is:

1. A stent comprising a plurality of stent struts forming at least one stent module wherein said stent module defines a channel and wherein at least one of said plurality of stent struts comprises a streamlined inner surface.

2. A stent according to claim 1 wherein at least one of said plurality of stent struts further comprises an inner leading surface and an inner trailing surface and wherein said inner leading surface and said inner trailing surface are asymmetrical.

3. A stent according to claim 2 wherein said inner leading surface comprises an inner leading curve having an inner leading curve radius and wherein said inner trailing surface comprises an inner trailing curve having an inner trailing curve radius and wherein said inner leading curve radius is larger than said inner trailing curve radius.

4. A stent according to claim 3 wherein said inner trailing surface is tapered.

5. A stent according to claim 4 wherein said inner surface has a shape selected from the group consisting of an airfoil and a teardrop.

6. A stent according to claim 2 wherein at least one of said plurality of stent struts further comprises an outer surface generally opposite of said inner surface and wherein said outer surface comprises an outer leading surface and an outer trailing surface and wherein at least one of said outer leading surface and said outer trailing surface are chamfered.

7. A stent according to claim 3 wherein at least one of said plurality of stent struts is formed from a material selected from the group consisting of metals, alloys, biologically compatible polymers, and combinations thereof.

8. A stent according to claim 7 wherein said inner surface of at least one of said plurality of stent struts is formed from at least one biologically compatible polymer.

9. A stent according to claim 1 wherein said stent comprises a plurality of stent modules.

10. A method of treating an anatomical passageway constriction comprising positioning at said constriction a stent comprising a plurality of stent struts forming at least one stent module wherein said stent module defines a channel and wherein at least one of said plurality of stent struts comprises a streamlined inner surface.

11. A method according to claim 10 wherein at least one of said plurality of stent struts further comprises an inner leading surface and an inner trailing surface and wherein said inner leading surface and said inner trailing surface are asymmetrical.

12. A method according to claim 11 wherein said inner leading surface comprises an inner leading curve having an inner leading curve radius and wherein said inner trailing surface comprises an inner trailing curve having an inner trailing curve radius and wherein said inner leading curve radius is larger than said inner trailing curve radius.

13. A method according to claim 12 wherein said inner trailing surface is tapered.

14. A method according to claim 13 wherein said inner surface has a shape selected from the group consisting of an airfoil and a teardrop.

15. A method according to claim 11 wherein at least one of said plurality of stent struts further comprises an outer surface generally opposite of said inner surface wherein said outer surface comprises an outer leading surface and an outer trailing surface wherein at least one of said outer leading surface and said outer trailing surface are chamfered.

16. A method according to claim 12 wherein at least one of said plurality of stent struts is formed from a material selected from the group consisting of metals, alloys, biologically compatible polymers, and combinations thereof.

17. A method according to claim 16 wherein said inner surface of at least one of said plurality of stent struts is formed from at least one biologically compatible polymer.

18. A method according to claim 10 wherein said stent comprises a plurality of stent modules.
